(19) United States
(12) Patent Application Publication
(10) Pub. No.: US 2024/0233918 A9
(43) Pub. Date: Jul. 11, 2024

(54) SYSTEM AND METHOD OF ADJUSTING DENTAL APPLIANCES USING ADDITIVE MANUFACTURING

(71) Applicant: SprintRay, Inc., Los Angeles, CA (US)

(72) Inventors: Amir Mansouri, Los Angeles, CA (US); Huijian Tian, Los Angeles, CA (US); Shukun Ye, Los Angeles, CA (US)

(21) Appl. No.: 18/382,818

(22) Filed: Oct. 23, 2023

Related U.S. Application Data
(60) Provisional application No. 63/418,422, filed on Oct. 21, 2022.

(51) Int. Cl. G16H 30/40; A61C 7/00

(52) U.S. Cl. CPC G16H 30/40; A61C 7/002

(57) ABSTRACT

A system and method of adjusting a dental appliance by modifying a corresponding digital dental appliance model. The system includes an additive manufacturing system (e.g., a 3D printer system) and a controller running software. The system is designed to (i) 3D print a dental appliance using an initial digital dental appliance model, (ii) determine any portions of the printed dental appliance that may require modification, (iii) automatically adjust the appliance's digital model by performing pixel offsetting to implement a modification, and (iv) reprint the appliance using the modified digital model.

### FIELD OF THE INVENTION

This invention relates to dental appliances, including a system and method of adjusting the fit of dental appliances using additive manufacturing.

### BACKGROUND

The conventional process of manufacturing dental appliances includes first taking an impression of a patient's teeth and then using the impression to create a physical model that may be used to create the appliance. The impression may be performed, and a model may be created in a dentist office by a dental professional. However, the impression and/or model must then be sent to an offsite dedicated dental laboratory to be used to create the corresponding dental appliance.

Once the appliance has been created, it may be sent back to the dentist office to be fit tested with the patient. However, if the appliance does not fit properly, e.g., if it is too loose or too tight, the process must be repeated starting with a new impression being performed, a new model being created and sent to the offsite laboratory, and a new appliance being made offsite and sent back to the dentist. As such, this process may be overlay expensive and time consuming.

Accordingly, there is a need for a system and method that manufactures a dental appliance onsite at a dentist office using additive manufacturing and a digital model of the appliance, that enables automatic modification of the digital model upon a determination that the dental appliance does not fit properly, and that reprints the appliance using the newly modified digital model.

### SUMMARY

According to one aspect, one or more embodiments are provided below for a system and method of adjusting dental appliances using additive manufacturing.

In one embodiment, a method of adjusting a dental appliance comprises determining if a first dental appliance provides a proper fit with a patient, in response to a determination that the first dental appliance does not provide a proper fit with the patient, then, by one or more computer systems, receiving a first digital dental appliance model corresponding to the first dental appliance, by one or more computer systems, turning off and/or turning on one or more pixels at a first side of the first digital dental appliance model to modify a size of the first digital dental appliance model, and by one or more computer systems, storing the modified digital dental appliance model as a second digital dental appliance model.

In another embodiment, the method comprises using an additive manufacturing system and the second digital dental appliance model to manufacture a second dental appliance.

In another embodiment, the turning off and/or turning on one or more pixels at a first side of the first digital dental appliance model includes turning off and/or turning on one or more pixels at a most outer outline of the first digital dental appliance model.

In another embodiments, the turning off one or more pixels at the most outer outline of the first digital dental appliance model results in a reduction in a size of the first digital dental appliance model.

In another embodiment, the reduction in the size of the first digital dental appliance model is distributed proportionally across an entirety of the digital dental appliance model.

In another embodiment, the turning on one or more pixels at the most outer outline of the first digital dental appliance model results in an increase in a size of the first digital dental appliance model.

In another embodiment, the increase in the size of the first digital dental appliance model is distributed proportionally across an entirety of the digital dental appliance model.

In another embodiment, the turning off and/or turning on one or more pixels at a first side of the first digital dental appliance model includes turning off and/or turning on one or more pixels at an outline of a design feature within the first digital dental appliance model.

In another embodiment, the turning off one or more pixels at the outline of the design feature within the first digital dental appliance model increases a size of the design feature within the first digital dental appliance model.

In another embodiment, the turning on one or more pixels at the outline of the design feature within the first digital dental appliance model decreases a size of the design feature within the first digital dental appliance model.

The presently disclosed orthodontic bracket and bracket support system and its method of manufacture and use is more fully described in the detailed description below.

### BRIEF DESCRIPTION OF THE DRAWINGS

Other objects, features, and characteristics of the present invention as well as the methods of operation and functions of the related elements of structure, and the combination of parts and economies of manufacture, will become more apparent upon consideration of the following description and the appended claims with reference to the accompanying drawings, all of which form a part of this specification. None of the drawings are to scale unless specifically stated otherwise.

FIG. 1 shows a dental appliance adjustment system in accordance with exemplary embodiments hereof;

FIG. 2 shows aspects of a digital dental appliance model in accordance with exemplary embodiments hereof;

FIG. 3 shows aspects of a digital dental appliance model in accordance with exemplary embodiments hereof;

FIG. 4 shows aspects of a digital dental appliance model in accordance with exemplary embodiments hereof;

FIG. 5 shows aspects of a digital dental appliance model in accordance with exemplary embodiments hereof;

FIG. 6 shows aspects of a digital dental appliance model in accordance with exemplary embodiments hereof;

FIG. 7 shows aspects of a digital dental appliance model in accordance with exemplary embodiments hereof;

FIG. 8 shows aspects of a digital dental appliance model in accordance with exemplary embodiments hereof;

FIG. 9 shows aspects of a digital dental appliance model in accordance with exemplary embodiments hereof;

FIG. 10 shows aspects of a graphical user interface (GUI) in accordance with exemplary embodiments hereof;

FIG. 11 shows actions that a dental appliance adjustment system may take in accordance with exemplary embodiments hereof;

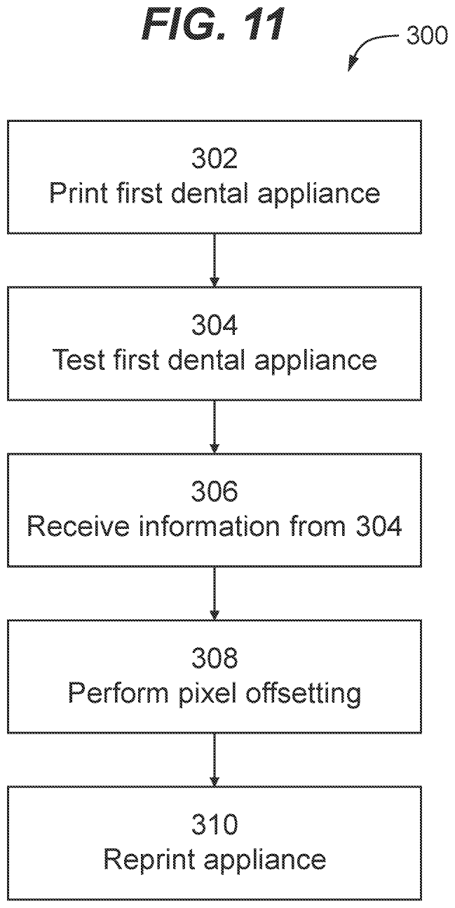

FIG. 12 shows actions that a dental appliance adjustment system may take in accordance with exemplary embodiments hereof; and

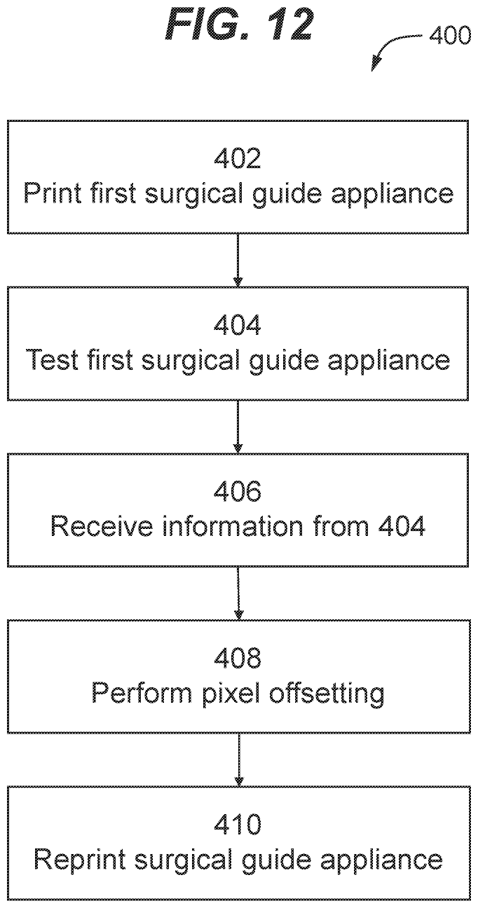

FIG. 13 depicts aspects of computing and computer devices in accordance with exemplary embodiments hereof.

### DETAILED DESCRIPTION OF EXEMPLARY EMBODIMENTS

In general, the system and method according to exemplary embodiments hereof includes a system and method of adjusting the fit of dental appliances using additive manufacturing. For example, the system and method may perform the following: (i) three-dimensionally print a dental appliance using an initial digital dental appliance model, (ii) determine any portions of the printed dental appliance that may require modification (e.g., any areas of the dental appliance that do not properly fit onto the patient's teeth), (iii) automatically adjust the appliance's digital model to implement a modification (resulting in a modified digital dental appliance model), and (iv) reprint the appliance using the modified digital model. The system and method also may provide other functionalities as described herein.

In a first example, the system may three-dimensionally (3D) print a first dental appliance using a first digital model. The first appliance may then be physically tested for proper fit with the patient's teeth. If the first appliance does not fit properly, e.g., if the appliance is too tight and/or too loose, the system may receive information regarding this condition, and may use this information to automatically adjust the size of the appliance's digital model to correct the fit accordingly. The system may then reprint the appliance using the adjusted digital model and the newly printed appliance may be retested for proper fitting with the patient's teeth.

In another example, the system may 3D print a dental surgical guide appliance that includes one or more surgical guide holes. The surgical guide appliance may then be fit tested to confirm that the guide holes are the proper size and at the proper location and angle. If the guide holes are deemed to require modification, the system may receive information regarding this condition and may use the information to automatically adjust the surgical guide's digital model as necessary. The system may then use the adjusted digital model to reprint the surgical guide appliance and the newly printed appliance may be tested for proper fitting.

It is understood that the example use cases described above are meant for demonstration and are non-limiting.

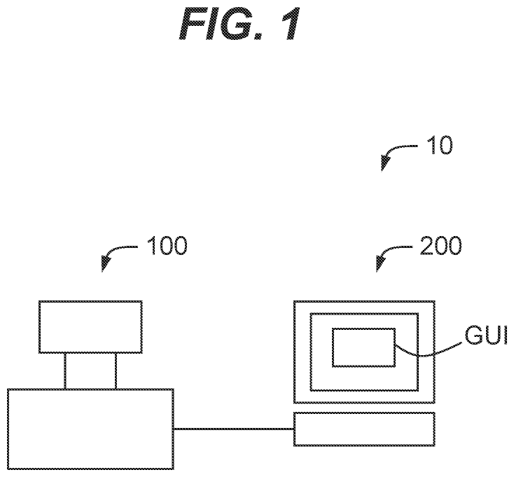

In some embodiments, as shown in FIG. 1, the dental appliance adjustment system 10 (also referred to herein as simply the system 10) includes an additive manufacturing system 100 (e.g., a 3D printing system) and a controller 200. The system 10 also may include additional elements as necessary for the system 10 to perform its functionalities.

For the purposes of this specification, the additive manufacturing system 100 will be described primarily as a 3D printer 100 (e.g., as a stereolithography (SLA) system). However, it is understood that any suitable type additive manufacturing system may be used and that the scope of the system 10 is not limited in any way by the type of additive manufacturing system(s) that it may employ.

In some embodiments, the controller 200 includes a computing device running software as described in other sections.

In some embodiments, the system 10 may use its controller 200 to turn on and/or turn off one or more pixels to the digital dental appliance model in the X- and/or Y-plane to modify the size of the model (e.g., its width and/or length) while maintaining the model's design details and overall geometry. This may be referred to as pixel offsetting.

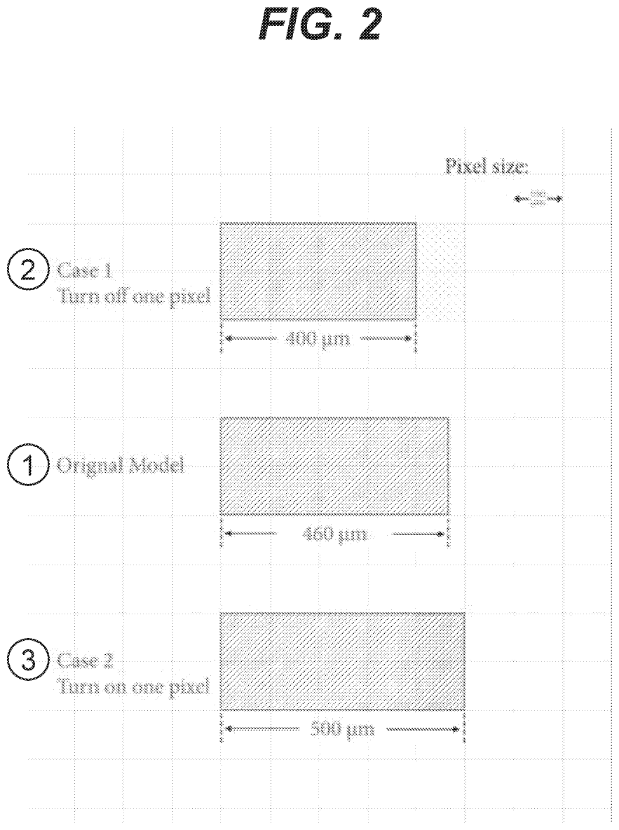

This concept is illustrated in FIG. 2 with the digital dental appliance models each represented as a simple block for demonstration. In this example, an initial dental appliance is formed using an initial digital dental appliance model (1). As shown, the width of the initial model (1) is 460 μm. The background grid represents an example grid of pixels with each pixel being 100 μm square.

In this example, if a first printed dental appliance produced from the first digital dental model (1) is deemed to be too large (e.g., too loose when fitted onto the patient's teeth), the system 10 may turn off one (or more) pixels at the outline of the first digital dental model (1) to create a second digital dental model (2) of reduced size. In this example, the pixel at the far-right outline of the first model (1) has been turned off resulting in its width being reduced by 60 μm. The modified model is then saved as a second digital model (2) with a width of 400 μm. The system 10 may then print a second dental appliance of reduced size using the second digital dental model (2).

In another example, if the first printed dental appliance is deemed to be too small (e.g., too tight when fitted onto the patient's teeth), the system 10 may turn on one or more pixels at the outline of the first digital dental model (1) to create a third digital dental model (3) of increased size. In this example, the pixel at the far-right outline of the first model (1) has been turned on resulting in its width being increased by 40 μm. The modified model is then saved as a third digital model (3) with a width of 500 μm. The system 10 may then print a third dental appliance of increased size using the third digital dental model (3).

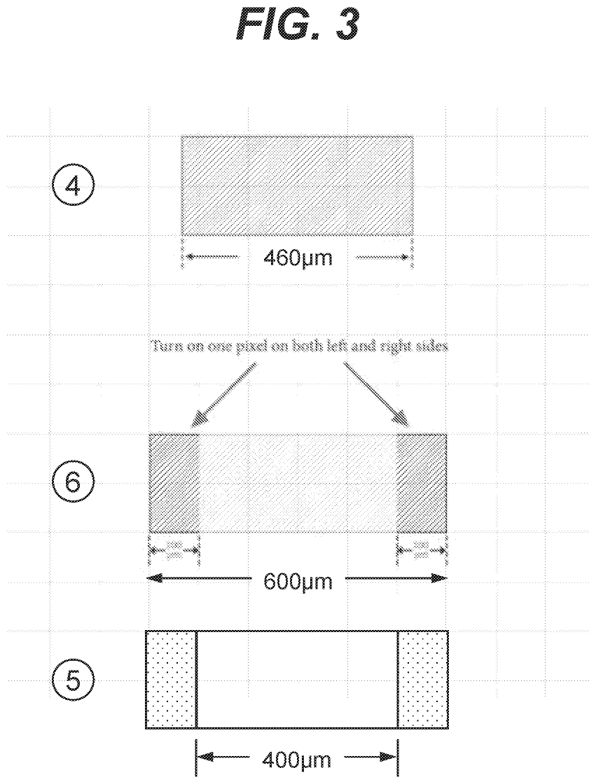

FIG. 3 shows a similar example but with the system 10 turning on and/or off one (or more) pixels on each of the digital appliance model's opposite sides (e.g., on the left and on the right). In this example, the width of the initial digital dental appliance model (4) is 460 μm and the background grid represents an example grid of pixels with each pixel being 100 μm square.

In this example, if a fourth printed dental appliance produced from the fourth digital dental appliance model (4) is deemed to be too large, the system 10 may turn off one (or more) pixels on both opposite sides (e.g., at the far-left outline and at the far-right outline) of the fourth digital dental model (4) to create a fifth digital dental model (5) of reduced size. In this example, pixels at both the far-left outline and at the far-right outline of the fourth digital model (4) have been turned off resulting in its width being reduced by 30 μm on each side (60 μm total). The modified model is then saved as a fifth digital model (5) with a width of 400 μm. The system 10 may then print a fifth dental appliance of increased size using the fifth digital dental model (5).

In another example, if the fourth printed dental appliance is deemed to be too small (e.g., too tight when fitted onto the patient's teeth), the system 10 may turn on one (or more) pixels on both opposite sides (e.g., at the far-left outline and at the far-right outline) of the fourth digital dental model (4) to create a sixth digital dental model (6) of increased size. In this example, pixels at both the far-left outline and at the far-right outline of the fourth digital model (4) have been turned on resulting in its width being increased by 70 μm on each side (140 μm total). The modified model is then saved as a sixth digital model (6) with a width of 600 μm. The system 10 may then print a sixth dental appliance of increased size using the sixth digital dental model (6).

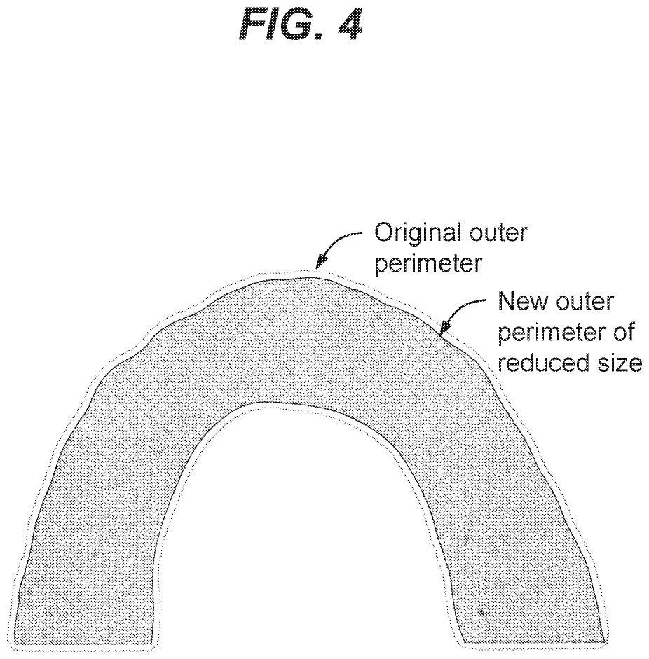

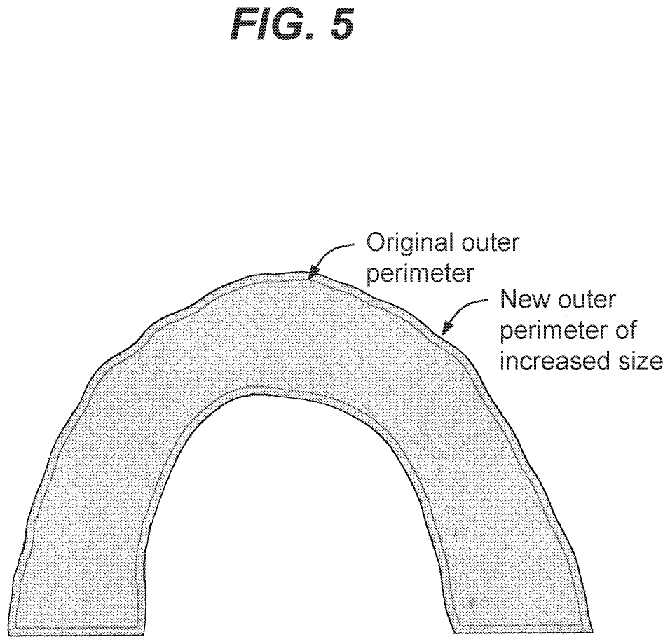

FIGS. 4-5 show the above concept applied to an exemplary dental appliance. As shown in FIG. 4, the system 10 may turn off an outer perimeter of one or more pixels about the entire perimeter (outer outline) of the digital appliance model to decrease the overall size of the resulting appliance, and as shown in FIG. 5, the system 10 may turn on an outer perimeter of one or more pixels about the entire perimeter (outer outline) of the digital appliance model to increase the overall size of the resulting appliance.

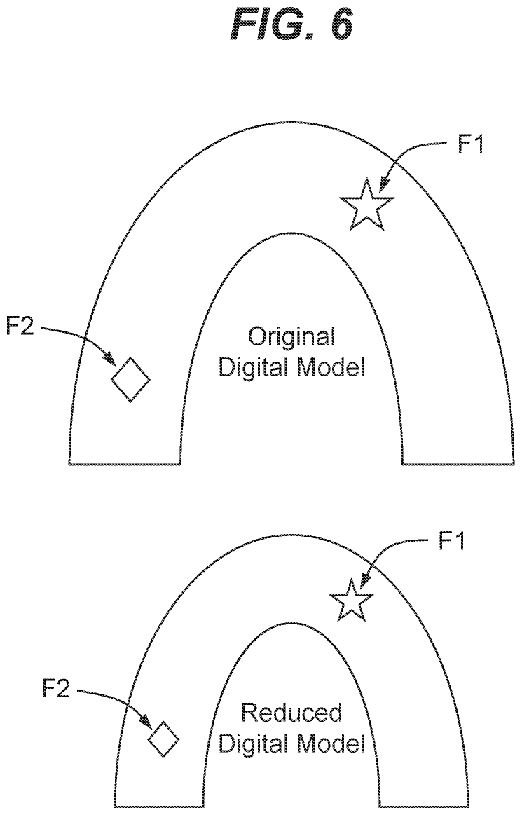

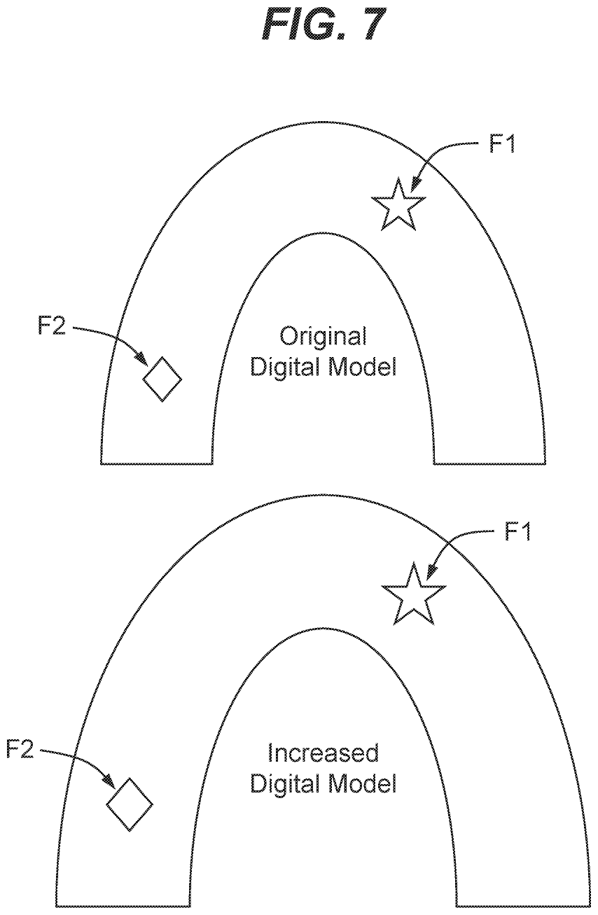

In some embodiments, as illustrated in FIGS. 6-7, a decrease or increase in size of an appliance caused by the system 10 turning off or turning on one or more pixels, respectively, may be distributed proportionally across the entire appliance. In this way, the size of one or more design features of the digital appliance model (e.g., individual recesses designed for each tooth) also may be resized proportionally. For example, FIG. 6 shows an initial digital dental appliance model with a first design feature F1 (depicted as a star) and a second design feature F2 (depicted as a diamond). FIG. 6 also shows a version of the original digital model that has been reduced in size by the system 10 using the pixel offsetting technique as described herein (i.e., by turning off one or more pixels). As shown, the first and second design features F1, F2 have each been reduced in size by a corresponding amount proportional to the overall amount of the decrease in the size of the overall appliance.

In another example, FIG. 7 shows the initial digital dental appliance model with the first and second design features F1, F2 followed by a version of the original digital model that has been increased in size by the system 10 using the pixel offsetting technique as described herein (i.e., by turning on one or more pixels). As shown, the first and second design features F1, F2 have each been increased in size by a corresponding amount proportional to the overall amount of the increase in the size of the overall appliance.

In some embodiments, the system 10 enables a user to lock one or more design features (e.g., F1 and/or F2 of FIGS. 6-7) of a digital dental appliance model such that the size of the locked feature(s) may not be affected by the overall decrease and/or increase of the appliance model by the system 10. In this case, locked features may not be resized while unlocked features may be resized proportionally. For example, a user may wish to resize the overall dental appliance including its respective teeth recesses but may not wish to resize one or more surgical guide holes located within the dental appliance. In this case, the user may simply lock the one or more surgical guide holes such that the guide holes are not resized when the system 10 resizes the overall appliance using pixel offsetting as described herein.

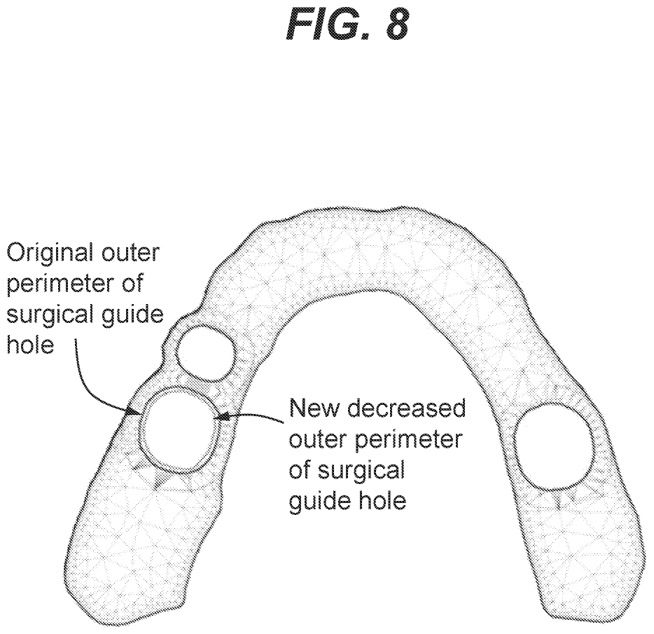

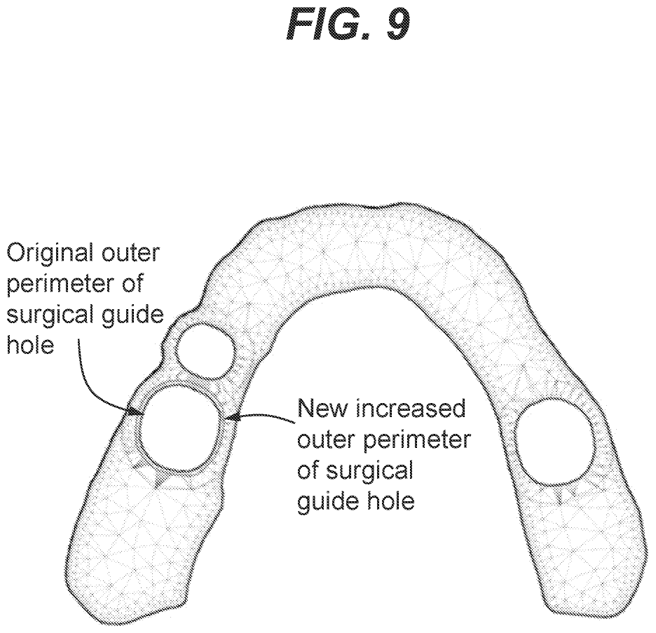

In some embodiments, as shown in FIGS. 8-9, the system 10 enables a user to choose a particular design feature of the digital dental appliance model to be resized. For example, a user may use the system 10 to resize a surgical guide hole within the interior of the dental model. As shown in FIG. 8, the system 10 may decrease the size of the guide hole by turning on one or more pixels about the hole's circumferential edge (its outline), and as shown in FIG. 9, the system 10 may increase the size of the guide hole by turning off one or more pixels about the hole's circumferential edge (its outline). In some embodiments, the user may choose for the system 10 to not affect the size of any other elements of the digital appliance model including its overall size while resizing the chosen guide hole(s), while in other embodiments, a user may choose for the system 10 to resize additional design features and/or the overall appliance model proportionally.

In all of the embodiments described herein, the system 10 may resize a digital dental appliance model, and/or any chosen aspect of the digital dental appliance model while preserving the model's overall design details (e.g., the teeth recesses, surgical guide holes, etc.) such that the accuracy of the digital appliance model is not negatively compromised by the modifications.

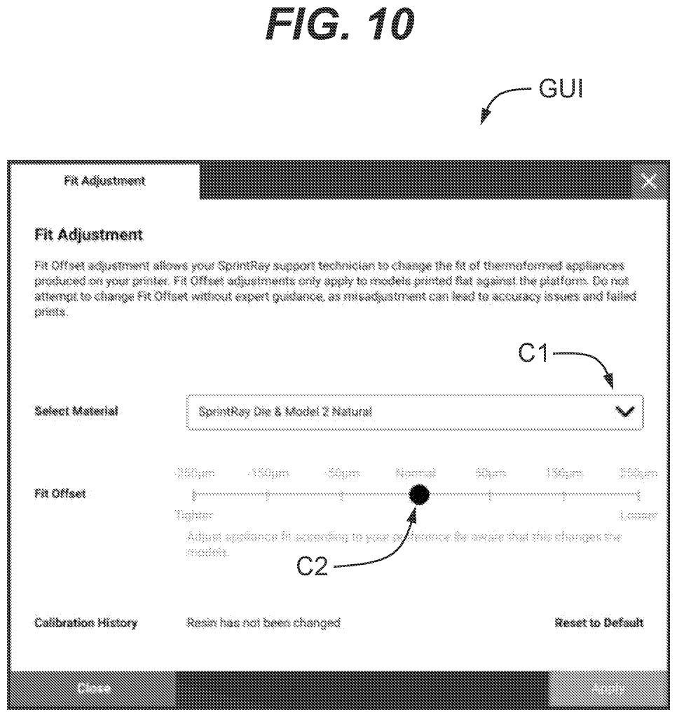

In some embodiments, as shown in FIG. 10, the system 10 includes a graphical user interface GUI to facilitate the resizing of one or more aspects of a digital dental appliance model (the pixel offsetting). In some embodiments, the GUI may include one or more control elements to enable the user to perform the desired modifications to the digital model(s). For example, the GUI may include a first control element C1 (e.g., a drop-down menu) to choose the type of resin material being used to print the appliance. This information may enable the system 10 to determine various characteristics of the appliance, e.g., the sizing of the one or more pixels that may be turned on or off during the modification process. In another example, the GUI also may include a second control element C2 (e.g., a slider) to choose the amount of decreasing and/or increasing of the appliance model to apply (e.g., the number of pixels for the system 10 to turn off and/or to turn on). In some embodiments, the GUI also may include one or more input fields for the user to enter the various information and/or any other types of GUI control elements as desired.

In some embodiments, the system 10 may perform the following actions 300 to modify a digital dental appliance model and its resulting printed dental appliance:

At 302, the system 10 may three-dimensionally (3D) print a first dental appliance using a first digital dental appliance model.

At 304, the first appliance may be physically tested for proper fit with the patient's teeth.

At 306, if it is deemed that the first appliance does not fit properly, e.g., if the appliance is too tight and/or too loose, the system 10 may receive information regarding this condition.

At 308, the system 10 may use the information from 306 to automatically adjust the size of the appliance's digital model to correct the fit accordingly. This may include performing pixel offsetting as described herein, i.e., the system 10 may turn off and/or may turn on one or more pixels (e.g., at the outline of the digital dental appliance model).

At 310, the system 10 may reprint the appliance using the adjusted digital model and the newly printed appliance may be retested for proper fitting with the patient's teeth.

In some embodiments, the system 10 may perform the following actions 400 to modify a digital surgical guide appliance model and its resulting printed surgical guide appliance:

At 402, the system 10 may three-dimensionally (3D) print a first dental surgical guide appliance that includes one or more surgical guide holes using a first digital surgical guide appliance model.

At 404, the surgical guide holes within the first surgical guide appliance may be physically tested for proper size, location, and angle, as well as proper fit with the patient's teeth.

At 406, if it is deemed that a surgical guide hole requires modification, e.g., if the guide hole is too small and/or too large, the system 10 may receive information regarding this condition.

At 408, the system 10 may use the information from 406 to automatically adjust the size of the guide hole accordingly. This may include performing pixel offsetting as described herein, i.e., the system 10 may turn off and/or may turn on one or more pixels (e.g., at the outline or edge of the guide hole within the digital surgical guide appliance model).

At 410, the system 10 may reprint the surgical guide appliance using the adjusted digital model and the newly printed appliance may be retested for proper size and fitting with the patient's teeth.

It is understood that any aspect or element of any embodiment described herein may be combined with any other aspect or element of any other embodiment to form additional embodiments of the system 10, all of which are within the scope of the system 10.

Computing

The services, mechanisms, operations, and acts shown and described above are implemented, at least in part, by software running on one or more computers or computer systems or devices. It should be appreciated that each user device is, or comprises, a computer system.

Programs that implement such methods (as well as other types of data) may be stored and transmitted using a variety of media (e.g., computer readable media) in a number of manners. Hard-wired circuitry or custom hardware may be used in place of, or in combination with, some or all of the software instructions that can implement the processes of various embodiments. Thus, various combinations of hardware and software may be used instead of software only.

One of ordinary skill in the art will readily appreciate and understand, upon reading this description, that the various processes described herein may be implemented by, e.g., appropriately programmed general purpose computers, special purpose computers and computing devices. One or more such computers or computing devices may be referred to as a computer system.

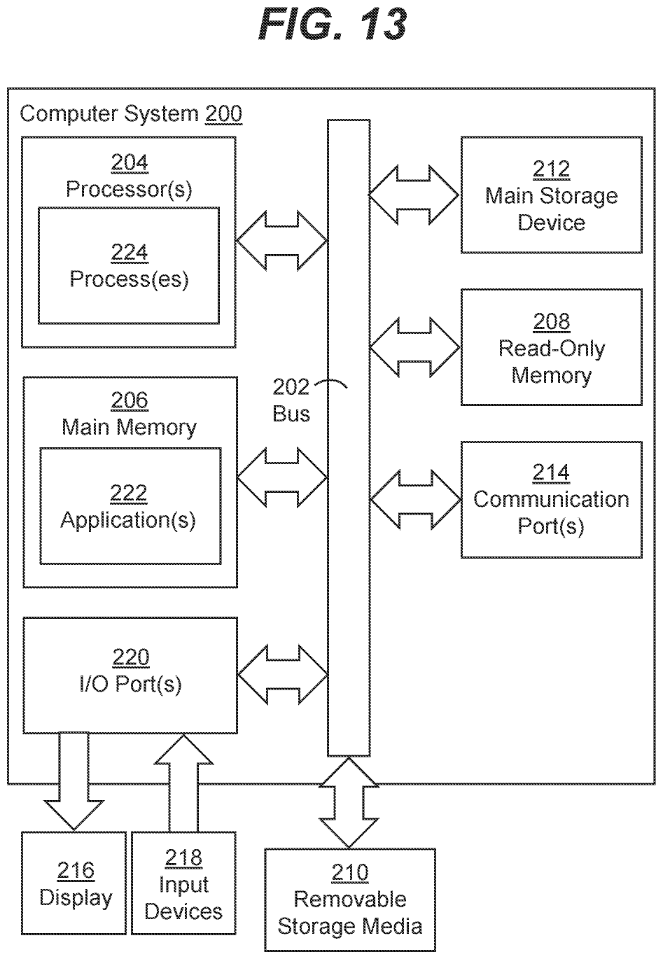

FIG. 13 is a schematic diagram of a computer system 200 upon which embodiments of the present disclosure may be implemented and carried out.

According to the present example, the computer system 200 includes a bus 202 (i.e., interconnect), one or more processors 204, one or more communications ports 214, a main memory 210, removable storage media 210, read-only memory 208, and a mass storage 212. Communication port(s) 214 may be connected to one or more networks by way of which the computer system 200 may receive and/or transmit data.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input devices and output devices that are appropriate to perform the process.

Processor(s) 204 can be (or include) any known processor, such as, but not limited to, an Intel® Itanium® or Itanium 2® processor(s), AMD® Opteron® or Athlon MP® processor(s), or Motorola® lines of processors, and the like. Communications port(s) 214 can be any of an RS-232 port for use with a modem-based dial-up connection, a 10/100 Ethernet port, a Gigabit port using copper or fiber, or a USB port, and the like. Communications port(s) 214 may be chosen depending on a network such as a Local Area Network (LAN), a Wide Area Network (WAN), a CDN, or any network to which the computer system 1600 connects. The computer system 200 may be in communication with peripheral devices (e.g., display screen 210, input device(s) 218) via Input/Output (I/O) port 220. Some or all of the peripheral devices may be integrated into the computer system 200, and the input device(s) 218 may be integrated into the display screen 210 (e.g., in the case of a touch screen).

Main memory 210 can be Random Access Memory (RAM), or any other dynamic storage device(s) commonly known in the art. Read-only memory 208 can be any static storage device(s) such as Programmable Read-Only Memory (PROM) chips for storing static information such as instructions for processor(s) 204. Mass storage 212 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 202 communicatively couples processor(s) 204 with the other memory, storage and communications blocks. Bus 202 can be a PCI/PCI-X, SCSI, a Universal Serial Bus (USB) based system bus (or other) depending on the storage devices used, and the like. Removable storage media 210 can be any kind of external hard-drives, floppy drives, IOMEGA® Zip Drives, Compact Disc-Read Only Memory (CD-ROM), Compact Disc—Re-Writable (CD-RW), Digital Versatile Disk-Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) which may be read by a computer, a processor, or a like device. Such a medium may take many forms, including but not limited to, non-volatile media, volatile media, and transmission media. Non-volatile media include, for example, optical or magnetic disks and other persistent memory. Volatile media include dynamic random-access memory, which typically constitutes the main memory of the computer. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

Various forms of computer readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards or protocols; and/or (iv) encrypted in any of a variety of ways well known in the art.

A computer-readable medium can store (in any appropriate format) those program elements that are appropriate to perform the methods.

As shown, main memory 210 is encoded with application(s) 222 that support(s) the functionality as discussed herein (an application 222 may be an application that provides some or all of the functionality of one or more of the mechanisms described herein). Application(s) 222 (and/or other resources as described herein) can be embodied as software code such as data and/or logic instructions (e.g., code stored in the memory or on another computer readable medium such as a disk) that supports processing functionality according to different embodiments described herein.

During operation of one embodiment, processor(s) 204 accesses main memory 210 via the use of bus 202 in order to launch, run, execute, interpret or otherwise perform the logic instructions of the application(s) 222. Execution of application(s) 222 produces processing functionality of the service(s) or mechanism(s) related to the application(s). In other words, the process(es) 224 represents one or more portions of the application(s) 222 performing within or upon the processor(s) 204 in the computer system 200.

It should be noted that, in addition to the process(es) 224 that carries(carry) out operations as discussed herein, other embodiments herein include the application 222 itself (i.e., the un-executed or non-performing logic instructions and/or data). The application 222 may be stored on a computer readable medium (e.g., a repository) such as a disk or in an optical medium. According to other embodiments, the application 222 can also be stored in a memory type system such as in firmware, read only memory (ROM), or, as in this example, as executable code within the main memory 210 (e.g., within Random Access Memory or RAM). For example, application 222 may also be stored in removable storage media 210, read-only memory 208, and/or mass storage device 212.

Those skilled in the art will understand that the computer system 200 can include other processes and/or software and hardware components, such as an operating system that controls allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, which can include hardware, software, firmware, or any combination thereof.

One of ordinary skill in the art will readily appreciate and understand, upon reading this description, that embodiments of an apparatus may include a computer/computing device operable to perform some (but not necessarily all) of the described process.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In another embodiment, the process includes some human intervention (e.g., a step is performed by or with the assistance of a human).

As used in this description, the term “portion” means some or all. So, for example, “A portion of X” may include some of “X” or all of “X”. In the context of a conversation, the term “portion” means some or all of the conversation.

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

As used herein, including in the claims, a list may include only one item, and, unless otherwise stated, a list of multiple items need not be ordered in any particular manner. A list may include duplicate items. For example, as used herein, the phrase “a list of XYZs” may include one or more “XYZs”.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

No ordering is implied by any of the labeled boxes in any of the flow diagrams unless specifically shown and stated. When disconnected boxes are shown in a diagram the activities associated with those boxes may be performed in any order, including fully or partially in parallel.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of adjusting a dental appliance comprising:
determining if a first dental appliance provides a proper fit with a patient;
in response to a determination that the first dental appliance does not provide a proper fit with the patient, then;
by one or more computer systems, receiving a first digital dental appliance model corresponding to the first dental appliance;
by one or more computer systems, turning off and/or turning on one or more pixels at a first side of the first digital dental appliance model to modify a size of the first digital dental appliance model; and
by one or more computer systems, storing the modified digital dental appliance model as a second digital dental appliance model.

2. The method of claim 1 further comprising:
using an additive manufacturing system and the second digital dental appliance model to manufacture a second dental appliance.

3. The method of claim 1 wherein the turning off and/or turning on one or more pixels at a first side of the first digital dental appliance model includes turning off and/or turning on one or more pixels at a most outer outline of the first digital dental appliance model.

4. The method of claim 3 wherein the turning off one or more pixels at the most outer outline of the first digital dental appliance model results in a reduction in a size of the first digital dental appliance model.

5. The method of claim 4 wherein the reduction in the size of the first digital dental appliance model is distributed proportionally across an entirety of the digital dental appliance model.

6. The method of claim 3 wherein the turning on one or more pixels at the most outer outline of the first digital dental appliance model results in an increase in a size of the first digital dental appliance model.

7. The method of claim 6 wherein the increase in the size of the first digital dental appliance model is distributed proportionally across an entirety of the digital dental appliance model.

8. The method of claim 1 wherein the turning off and/or turning on one or more pixels at a first side of the first digital dental appliance model includes turning off and/or turning on one or more pixels at an outline of a design feature within the first digital dental appliance model.

9. The method of claim 8 wherein the turning off one or more pixels at the outline of the design feature within the first digital dental appliance model increases a size of the design feature within the first digital dental appliance model.

10. The method of claim 8 wherein the turning on one or more pixels at the outline of the design feature within the first digital dental appliance model decreases a size of the design feature within the first digital dental appliance model.
